(19) United States
(12) Patent Application Publication
(10) Pub. No.: US 2010/0247533 A1
(43) Pub. Date: Sep. 30, 2010

(54) TREATMENT WITH A HUMANIZED ANTI-EGFR IgG1 ANTIBODY AND IRINOTECAN

(76) Inventors: Thomas Friess, Diessen-Dettenhofen (DE); Ekkehard Moessner, Kreuzlingen (CH); Samuel Moser, Zuerich (CH); Pablo Umana, Zuerich (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/748,506

(22) Filed: Mar. 29, 2010

(30) Foreign Application Priority Data
Mar. 31, 2009 (EP) 09156844.4

(51) Int. Cl. A61K 39/395; A61P 35/00

(52) U.S. Cl. 424/133.1

(57) ABSTRACT

The present invention provides a humanized anti-EGFR IgG1 antibody and irinotecan for combined use in treating cancer, with or without additional agents or treatments, such as other anti-cancer drugs or radiation therapy. The invention also encompasses a pharmaceutical composition that is comprised of a combination of a humanized anti-EGFR IgG1 antibody and irinotecan in a pharmaceutically acceptable carrier, and methods for the treatment of cancer comprising administering both irinotecan and a humanized anti-EGFR IgG1 antibody.

### PRIORITY TO RELATED APPLICATION(S)

This application claims the benefit of European Patent Application No. 09156844.4, filed Mar. 31, 2009, which is hereby incorporated by reference in its entirety.

The instant application contains a Sequence Listing in ASCII text which has been submitted via EFS-Web and is hereby incorporated by reference in its entirety.

The present invention is directed to agents and pharmaceutical compositions for use in treating cancer. In particular, the present invention is directed to a humanized anti-EGFR IgG1 antibody and irinotecan for combined use in the treatment of cancer.

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body.

A multitude of therapeutic agents have been developed over the past few decades for the treatment of various types of cancer. The most commonly used types of anticancer agents include: Microtubule disruptors (e.g. vinca alkaloids such as vinblastine or vincristine, taxanes such as docetaxel or paclitaxel, epothilones such as ixabepilone), antimetabolites (e.g. anti-folates such as methotrexate or aminopterin, anti-purines such as fludarabine, anti-pyrimidines such as fluorouracil, capecitabine or gemcitabine), topoisomerase inhibitors (e.g. camptothecin, irinotecan or etoposide), DNA intercalators (e.g. doxorubicin, daunorubicin, actinomycin, bleomycin), alkylating agents (e.g. cyclophosphamide, chlorambucil, carmustine, nimustine, streptozocin, busulfan, cisplatin, oxaliplatin, triethylenemelamine, dacarbazine) and hormonal therapy (e.g. glucocorticoids, aromatase inhibitors such as tamoxifene, antiandrogens such as flutamide, gonadotropin-releasing hormone (GnRH) analogs such as leuprolide).

Irinotecan (Campto®) is a topoisomerase I inhibitor. The substance is a semisynthetic analog of camptothecin, a natural alkaloid. It is used for the treatment of different types of cancer, e.g. colon cancer, often in combination with other chemotherapeutic agents.

More recently, the importance of targeted therapies in cancer therapy has grown. Such substances—either small molecules or biotherapeutics such as antibodies—interfere with specific targets, e.g. cell surface receptors known to promote carcinogenesis and tumor growth.

### Epidermal Growth Factor Receptor (EGFR) and Anti-EGFR Antibodies

Human epidermal growth factor receptor (also known as HER-1 or ErbB-1, and referred to herein as “EGFR”) is a 170 kDa transmembrane receptor encoded by the c-erbB protooncogene, and exhibits intrinsic tyrosine kinase activity (Modjtahedi et al., Br J Cancer 73, 228-235 (1996); Herbst and Shin, Cancer 94, 1593-1611 (2002)). SwissProt database entry number P00533 provides the sequence of EGFR. There are also isoforms and variants of EGFR (e.g., alternative RNA transcripts, truncated versions, polymorphisms, etc.) including but not limited to those identified by SwissProt database entry numbers P00533-1, P00533-2, P00533-3, and P00533-4. EGFR is known to bind ligands including epidermal growth factor (EGF), transforming growth factor-α (TGF-α), amphiregulin, heparin-binding EGF (HB-EGF), betacellulin, and epiregulin (Herbst and Shin, Cancer 94, 1593-1611 (2002); Mendelsohn and Baselga, Oncogene 19, 6550-6565 (2000)). EGFR regulates numerous cellular processes via tyrosine kinase-mediated signal transduction pathways, including, but not limited to, activation of signal transduction pathways that control cell proliferation, differentiation, cell survival, apoptosis, angiogenesis, mitogenesis, and metastasis (Atalay et al., Ann Oncology 14, 1346-1363 (2003); Tsao and Herbst, Signal 4, 4-9 (2003); Herbst and Shin, Cancer 94, 1593-1611 (2002); Modjtahedi et al., Br J Cancer 73, 228-235 (1996)).

Overexpression of EGFR has been reported in numerous human malignant conditions, including cancers of the bladder, brain, head and neck, pancreas, lung, breast, ovary, colon, prostate, and kidney (Atalay et al., Ann Oncology 14, 1346-1363 (2003); Herbst and Shin, Cancer 94, 1593-1611 (2002) Modjtahedi et al., Br. J. Cancer 73, 228-235 (1996)). In many of these conditions, the overexpression of EGFR correlates or is associated with poor prognosis of the patients (Herbst and Shin, Cancer 94, 1593-1611 (2002) Modjtahedi et al., Br J Cancer 73, 228-235 (1996)). EGFR is also expressed in the cells of normal tissues, particularly the epithelial tissues of the skin, liver, and gastrointestinal tract, although at generally lower levels than in malignant cells (Herbst and Shin, Cancer 94, 1593-1611 (2002)).

Various strategies to target EGFR and block EGFR signaling pathways have been reported. Small-molecule tyrosine kinase inhibitors like gefitinib, erlotinib, canertinib/CI-1033, pelitinib/EKB-569, neratinib/HKI-272, lapatinib/GW572016 and others block autophosphorylation of EGFR in the intracellular tyrosine kinase region, thereby inhibiting downstream signaling events (Tsao and Herbst, Signal 4, 4-9 (2003)). Monoclonal antibodies, on the other hand, target the extracellular portion of EGFR, which results in blocking ligand binding and thereby inhibits downstream events such as cell proliferation (Tsao and Herbst, Signal 4, 4-9 (2003)).

Several murine monoclonal antibodies have been generated which achieve such a block in vitro and which have been evaluated for their ability to affect tumor growth in mouse xenograft models (Masui et al., Cancer Res. 46, 5592-5598 (1986); Masui et al., Cancer Res 44, 1002-1007 (1984); Goldstein et al., Clin Cancer Res 1, 1311-1318 (1995)). For example, EMD 55900 (EMD Pharmaceuticals) is a murine anti-EGFR monoclonal antibody that was raised against the human epidermoid carcinoma cell line A431 and was tested in clinical studies of patients with advanced squamous cell carcinoma of the larynx or hypopharynx (Bier et al., Eur Arch Otohinolaryngol 252, 433-9 (1995)). In addition, the rat monoclonal antibodies ICR16, ICR62, and ICR80, which bind the extracellular domain of EGFR, have been shown to be effective at inhibiting the binding of EGF and TGF-α the receptor (Modjtahedi et al., Int J Cancer 75, 310-316 (1998)). The murine monoclonal antibody (mAb) 425 is another mAb that was raised against the human A431 carcinoma cell line and was found to bind to a polypeptide epitope on the external domain of the human epidermal growth factor receptor (Murthy et al., Arch Biochem Biophys 252, 549-560 (1987)). A potential problem with the use of murine antibodies in therapy is that non-human monoclonal antibodies can be recognized by the human host as foreign proteins; therefore, repeated injections of such antibodies can lead to the induction of immune responses leading to harmful hypersensitivity reactions. For murine monoclonal antibodies, this is often referred to as a Human Anti-Mouse Antibody, or “HAMA”, response, or a Human Anti-Rat Antibody, or “HARA”, response. Additionally, these “foreign” antibodies can be attacked by the immune system of the host such that they are, in effect, neutralized before they reach their target site. Furthermore, non-human monoclonal antibodies (e.g., murine monoclonal antibodies) typically lack human effector functionality, i.e., they are unable to, inter alia, mediate complement dependent lysis or lyse human target cells through antibody dependent cell-mediated toxicity or Fc-receptor mediated phagocytosis.

To circumvent these problems, chimeric, humanized or even fully human antibodies have been developed, in which only the variable domains, the complementarity determining regions (CDRs) or no parts at all, respectively, are of murine origin, while all other parts of the antibody, in particular the Fc region, are of human origin.

For example, IMC-C225/cetuximab (Erbitux®; ImClone) is a chimeric mouse/human anti-EGFR mAb (based on mouse M225 monoclonal antibody, which resulted in HAMA responses in human clinical trials) that has been reported to demonstrate antitumor efficacy in various human xenograft models (Goldstein et al., Clin Cancer Res 1, 1311-1318 (1995); Herbst and Shin, Cancer 94, 1593-1611 (2002)). The efficacy of IMC-C225 has been attributed to several mechanisms, including inhibition of cell events regulated by EGFR signaling pathways, and possibly by increased antibody-dependent cell-mediated cytotoxicity (ADCC) activity (Herbst and Shin, Cancer 94, 1593-1611 (2002)). IMC-C225 was also used in clinical trials, including in combination with radiotherapy and chemotherapy (Herbst and Shin, Cancer 94, 1593-1611 (2002)). Also, U.S. Pat. No. 5,891,996 (Mateo de Acosta del R10 et al.) discusses a mouse/human chimeric antibody, R3, directed against EGFR. A humanized, R3-based antibody, h-R3/nimotuzumab Mateo et al., Immunotechnology 3, 71-81 (1997); Crombet-Ramos et al., Int J Cancer 101, 567-575 (2002), Boland & Bebb, Expert Opin Biol Ther 9, 1199-1206 (2009), is being developed by Oncoscience (Wedel, Germany) for cancer therapy. U.S. Pat. No. 5,558,864 discusses generation of chimeric and humanized forms of the murine anti-EGFR monoclonal antibody (mAb) 425, and a humanized mAb 425-based antibody, EMD72000/matuzumab (Bier et al., Cancer Immunol Immunother 46, 167-173 (1998), Kim, Curr Opin Mol Ther 6, 96-103 (2004)), is being developed by Merck (Darmstadt, Germany) for cancer therapy. Abgenix, Inc. (Fremont, Calif.) develops ABX-EGF/panitumumab for cancer therapy. ABX-EGF is a fully human anti-EGFR mAb (Yang et al., Crit. Rev Oncol/Hematol 38; 17-23 (2001)). Another fully human anti-EGFR mAb, 2F8/zalutumumab, has been developed by Genmab Inc. (Princeton, N.J.) (Bleeker et al., J Immunol 173, 4699-4707 (2004), Lammerts van Bueren, PNAS105, 6109-6114 (2008)).

### Antibody Glycosylation

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions (Jenkins et al., Nature Biotechnol 14, 975-81 (1996)).

IgG1 type antibodies, the most commonly used antibodies in cancer immunotherapy, are glycoproteins that have a conserved N-linked glycosylation site at Asn 297 in each CH2 domain. The two complex biantennary oligosaccharides attached to Asn 297 are buried between the CH2 domains, forming extensive contacts with the polypeptide backbone, and their presence is essential for the antibody to mediate effector functions such as antibody dependent cell-mediated cytotoxicity (ADCC) (Lifely et al., Glycobiology 5, 813-822 (1995); Jefferis et al., Immunol Rev 163, 59-76 (1998); Wright and Morrison, Trends Biotechnol 15, 26-32 (1997)).

Cell-mediated effector functions of monoclonal antibodies, such as the anti-EGFR antibodies mentioned above (e.g. cetuximab, nimotuzumab, panitumumab), can be enhanced by engineering their oligosaccharide component as described in Umaña et al., Nat Biotechnol 17, 176-180 (1999) and U.S. Pat. No. 6,602,684 (WO 99/54342). Umana et al. showed that overexpression of β(1,4)-N-acetylglucosaminyltransferase III (GnTIII), a glycosyltransferase catalyzing the formation of bisected oligosaccharides, in Chinese hamster ovary (CHO) cells significantly increases the in vitro ADCC activity of antibodies produced in those cells. Alterations in the composition of the Asn 297 carbohydrate or its elimination also affect binding of the antibody Fc-domain to FcγR and C1q protein (Umana et al., Nat Biotechnol 17, 176-180 (1999); Davies et al., Biotechnol Bioeng 74, 288-294 (2001); Mimura et al., J Biol Chem 276, 45539-45547 (2001); Radaev et al., J Biol Chem 276, 16478-16483 (2001); Shields et al., J Biol Chem 276, 6591-6604 (2001); Shields et al., J Biol Chem 277, 26733-26740 (2002); Simmons et al., J Immunol Methods 263, 133-147 (2002)).

An anti-neoplastic drug would ideally kill cancer cells selectively, with a wide therapeutic index relative to its toxicity towards non-malignant cells. It would also retain its efficacy against malignant cells, even after prolonged exposure to the drug. Unfortunately, none of the current anti-cancer therapies possess such an ideal profile. Instead, most possess very narrow therapeutic indexes. Furthermore, cancerous cells exposed to slightly sub-lethal concentrations of an anti-neoplastic agent will very often develop resistance to such an agent, and quite often cross-resistance to several other antineoplastic agents as well.

Thus, there is a need for more efficacious treatment for neoplasia and other proliferative disorders. Strategies for enhancing the therapeutic efficacy of existing drugs have involved changes in the schedule for their administration, and also their use in combination with other anticancer or biochemical modulating agents. Combination therapy is well known as a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone). For example, when combined with 5-fluorouracil and leucovorin, oxaliplatin exhibits response rates of 25-40% as first-line treatment for colorectal cancer (Raymond, E. et al., Semin Oncol 25(2 Suppl. 5), 4-12 (1998)).

Likewise, the combined use of antibodies, directed to specific targets on the surface of cancer cells, with chemotherapeutic agents might increase anti-cancer efficacy, compared to treatment with a single agent.

### DESCRIPTION OF THE INVENTION

Recognizing the great therapeutic potential of combining antibodies which target surface receptors on cancer cells involved in cancer progression with chemotherapeutic agents, the present invention provides a humanized anti-EGFR IgG1 antibody and irinotecan for combined use in treating cancer.

The invention also encompasses a pharmaceutical composition, in particular for use in treating cancer, comprising a humanized anti-EGFR IgG1 antibody and irinotecan in a pharmaceutically acceptable carrier.

The present invention is further directed to a method for the treatment of cancer comprising administering to a subject in need a humanized anti-EGFR IgG1 antibody and irinotecan.

Preferably, a therapeutically effective amount of the combination of the humanized anti-EGFR IgG1 antibody and irinotecan is intended for administration to the patient simultaneously or sequentially, in the same or in different formulations and with or without additional agents or treatments, such as other anti-cancer drugs or radiation therapy.

Preferred humanized anti-EGFR IgG1 antibodies useful for the present invention are described in WO 2006/082515 and WO 2008/017963, the entire content of which is incorporated herein by reference, and include antibodies which are characterized in that they are chimeric antibodies having the binding specificity of the rat monoclonal antibody ICR62 and that their effector functions are enhanced by altered glycosylation.

Preferred anti-EGFR antibodies are characterized in that they comprise at least one (i.e. one, two, three, four, five, or six) complementarity determining region (CDR) of the rat ICR62 antibody, or a variant or truncated form thereof containing at least the specificity-determining residues for said CDR, and comprising a sequence derived from a heterologous polypeptide. By “specificity-determining residue” is meant those residues that are directly involved in the interaction with the antigen. Specifically, preferred anti-EGFR antibodies comprise: (a) a heavy chain CDR1 sequence selected from a group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:13; (b) a heavy chain CDR2 sequence selected from a group consisting of: SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30; and (c) the heavy chain CDR3 sequence SEQ ID NO:31. Preferred anti-EGFR antibodies further comprise: (a) a light chain CDR1 sequence selected from the group consisting of SEQ ID NO:32 and SEQ ID NO:33; (b) the light chain CDR2 sequence SEQ ID NO:34; and (c) the light chain CDR3 sequence SEQ ID NO:35.

More preferred anti-EGFR antibodies are characterized in that they comprise at least three CDRs of the rat ICR62 antibody, or variants or truncated forms thereof containing at least the specificity-determining residues for said CDRs.

Most preferred anti-EGRF antibodies useful for the present invention comprise:

a) in the heavy chain variable domain a CDR1 of SEQ ID NO:1, a CDR2 of SEQ ID NO:16, and a CDR3 of SEQ ID NO:31, and
b) in the light chain variable domain a CDR1 of SEQ ID NO:33, a CDR2 of SEQ ID NO:34, and a CDR3 of SEQ ID NO:35.

The possible CDR sequences of preferred anti-EGFR antibodies useful for the invention are summarized in Table 1 (heavy chain CDRs) and Table 2 (light chain CDRs).

TABLE 1
Heavy chain CDR amino acid sequences
of preferred anti-EGFR antibodies.*

| | | | SEQ |
|---|---|---|---|
| | | Amino Acid | ID |
| CDR | | Sequence | NO |
| Heavy | Kabat | DYKIH | 1 |
| Chain | | DYAIS | 2 |
| CDR1 | | DYYMH | 3 |
| | | DYKIS | 4 |
| | Chothia | GFTFTDY | 5 |
| | | GYTFTDY | 6 |
| | | GYSFTDY | 7 |
| | AbM | GFTFTDYKIH | 8 |
| | | GFTFTDYAIS | 9 |
| | | GFTFTDYYMH | 10 |
| | | GYTFTDYYMH | 11 |
| | | GYSFTDYKIH | 12 |
| | | GFTFTDYKIS | 13 |
| Heavy | Kabat | YFNPNSGYSTYNEKFKS | 14 |
| Chain | | GINPNSGYSTYAQKFQG | 15 |
| CDR2 | | YFNPNSGYSTYAQKFQG | 16 |
| | | WINPNSGYSTYAQKFQG | 17 |
| | | WINPNSGYSTYSPSFQG | 18 |
| | | WINPNSGYSTYNEKFQG | 19 |
| | | YFNPNSGYSNYAQKFQG | 20 |
| | | YFNPNSGYATYAQKFQG | 21 |
| | | YFNPNSGYSTYSPSFQG | 22 |
| | Chothia | NPNSGYST | 23 |
| | | NPNSGYSN | 24 |
| | | NPNSGYAT | 25 |
| | AbM | YFNPNSGYST | 26 |
| | | GINPNSGYST | 27 |
| | | WINPNSGYST | 28 |
| | | YFNPNSGYSN | 29 |
| | | YFNPNSGYAT | 30 |
| Heavy | Kabat | LSPGGYYVMDA | 31 |
| Chain | Chothia | | |
| CDR3 | AbM | | |

TABLE 2
Light chain CDR amino acid sequences
of preferred anti-EGFR antibodies.*

| | | SEQ ID |
|---|---|---|
| CDR | Amino Acid Sequence | NO |
| Kabat Light | KASQNINNYLN | 32 |
| Chain CDR1 | RASQGINNYLN | 33 |
| Kabat Light | NTNNLQT | 34 |
| Chain CDR2 | | |
| Kabat Light | LQHNSFPT | 35 |
| Chain CDR3 | | |

*“Kabat” refers to the CDRs as defined by Kabat et al., “Sequences of Proteins of Immunological Interest”, National Institutes of Health, Bethesda (1983)
“Chothia” refers to the CDRs as defined by Chothia et al., J Mol Biol 196, 901-917(1987)
“AbM” refers to the CDRs as defined by Oxford Molecular's AbM antibody modeling software

Preferred anti-EGFR antibodies useful for the invention have heavy and light chain variable domain framework sequences from a humanized immunoglobulin.

Other preferred anti-EGFR antibodies useful for the present invention comprise the heavy chain variable domain (V_{H}) of the rat ICR62 antibody according to SEQ ID NO:36, or a variant thereof; and a non-murine polypeptide. Further, preferred anti-EGFR antibodies may comprise the light chain variable domain (V_{L}) of the rat ICR62 antibody according to SEQ ID NO:37, or a variant thereof; and a non-murine polypeptide.

More preferred anti-EGFR antibodies useful for the invention comprise the heavy chain variable domain of SEQ ID NO:38 and the light chain variable domain of SEQ ID NO:39.

The heavy and light chain variable domain amino acid sequences of preferred anti-EGFR antibodies are shown in Table 3. The preferred anti-EGFR antibodies useful for the invention may also comprise amino acid sequences of at least 80%, 85%, 90%, 95%, 96%, 97%, 98% or 99% sequence identity to those shown in Table 3, or the amino acid sequences shown in Table 3 with conservative amino acid substitutions.

TABLE 3
Heavy and light chain variable domain amino
acid sequences of preferred anti-EGFR
antibodies.

| | | SEQ |
|---|---|---|
| | | ID |
| CONSTRUCT | AMINO ACID SEQUENCE | NO |
| ICR62 V_{H} | QVNLLQSGAALVKPGASVKLSCKGSGFTFTDYKIH | 36 |
| | WVKQSHGKSLEWIGYFNPNSGYSTYNEKFKSKATL | |
| | TADKSTDTAYMELTSLTSEDSATYYCTRLSPGGYY | |
| | VMDAWGQGASVTVSS | |
| ICR62 V_{L} | DIQMTQSPSFLSASVGDRVTINCKASQNINNYLNWY | 37 |
| | QQKLGEAPKRLIYNTNNLQTGIPSRFSGSGSGTDYT | |
| | LTISSLQPEDFATYFCLQHNSFPTFGAGTKLELKRT | |
| I-HHD V_{H} | QVQLVQSGAEVKKPGSSVKVSCKASGFTFTDYKIH | 38 |
| | WVRQAPGQGLEWMGYFNPNSGYSTYAQKFQGRV | |
| | TITADKSTSTAYMELSSLRSEDTAVYYCARLSPGGY | |
| | YVMDAWGQGTTVTVSS | |
| I-KC V_{L} | DIQMTQSPSSLSASVGDRVTITCRASQGINNYLNWY | 39 |
| | QQKPGKAPKRLIYNTNNLQTGVPSRFSGSGSGTEFT | |
| | LTISSLQPEDFATYYCLQHNSFPTFGQGTKLEIKRT | |

Preferred anti-EGFR antibodies useful for the invention are primatized or, more preferred, humanized antibodies.

Preferably, the anti-EGFR antibodies useful for the invention comprise a human Fc region. More preferably, the human heavy chain constant region is Ig gamma-1, as set forth in SEQ ID NO:40, i.e. the antibody is of human IgG1 subclass.

Human heavy chain constant region Ig gamma-1 amino acid sequence (SEQ ID NO:40): TKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKAEPKSCDKTHTCPPCPAPELLGGPS VFLFPPKPKDTLMISRTPEVTCVVVDVSHEDPEVKFNWYVDGVEVHNAKTKPREEQYN STYRVVSVLTVLHQDWLNGKEYKCKVSNKALPAPIEKTISKAKGQPREPQVYTLPPSRD ELTKNQVSLTCLVKGFYPSDIAVEWESNGQPENNYKTTPPVLDSDGSFFLYSKLTVDKS RWQQGNVFSCSVMHEALHNHYTQKSLSLSPGK

However, variants and isoforms of the human Fc region are also contemplated. For example, variant Fc regions suitable for use in the present invention can be produced according to the methods taught in U.S. Pat. No. 6,737,056 to Presta (Fc region variants with altered effector function due to one or more amino acid modifications); or in U.S. Patent Appl. Nos. 60/439,498; 60/456,041; 60/514,549; or WO 2004/063351 (variant Fc regions with increased binding affinity due to amino acid modification); or in U.S. patent application Ser. No. 10/672,280 or WO 2004/099249 (Fc variants with altered binding to FcγR due to amino acid modification), the contents of each of which is herein incorporated by reference in its entirety.

In another preferred embodiment, anti-EGFR antibodies useful for the invention have been glycoengineered to have an altered oligosaccharide structure in the Fc region.

Specifically, preferred anti-EGFR antibodies have an increased proportion of non-fucosylated oligosaccharides in the Fc region as compared to non-glycoengineered antibodies. Preferably, the percentage of non-fucosylated oligosaccharides is at least 20%, more preferably at least 50-70%, most preferably at least 75%. Anti-EGFR antibodies useful for the invention having such percentages of non-fucosylated oligosaccharides are further termed as partially fucosylated. The non-fucosylated oligosaccharides may be of the hybrid or complex type.

Preferred anti-EGFR antibodies may also have an increased proportion of bisected oligosaccharides in the Fc region. Preferably, the percentage of bisected oligosaccharides in the Fc region of the antibody is at least 50%, more preferably, at least 60%, at least 70%, at least 80%, or at least 90%, and most preferably at least 90-95% of the total oligosaccharides.

Particularly preferred anti-EGFR antibodies have an increased proportion of bisected, non-fucosylated oligosaccharides in the Fc region. The bisected, non-fucosylated oligosaccharides may be either hybrid or complex. Specifically, anti-EGFR antibodies are preferred in which at least 15%, more preferably at least 20%, more preferably at least 25%, more preferably at least 30%, more preferably at least 35% of the oligosaccharides in the Fc region of the antibody are bisected, non-fucosylated.

Preferred anti-EGFR antibodies are also characterized in that they have been glycoengineered to have increased effector function and/or increased Fc receptor binding affinity.

Preferably, the increased effector function is one or more of the following: increased Fc-mediated cellular cytotoxicity (including increased antibody-dependent cell-mediated cytotoxicity (ADCC)), increased antibody-dependent cellular phagocytosis (ADCP), increased cytokine secretion, increased immune-complex-mediated antigen uptake by antigen-presenting cells, increased binding to natural killer (NK) cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming. The increased Fc receptor binding affinity is preferably increased binding to a Fcγ activating receptor, most preferably increased binding to FcγRIIIa.

The most preferred anti-EGFR antibody useful for the invention is characterized in that it comprises the heavy chain variable domain of SEQ ID NO:38 and the light chain variable domain of SEQ ID NO:39, is humanized, and comprises the human heavy chain constant region Ig gamma-1, as set forth in SEQ ID NO:40. This antibody is termed “GlycArt-mAb”. GlycArt-mAb may or may not be partially fucosylated, i.e. glycoengineered as described above, to have an increased proportion of non-fucosylated oligosaccharides in the Fc region as compared to non-glycoengineered antibodies.

Techniques for the production and isolation of monoclonal antibodies and antibody fragments, methods for humanizing non-human antibodies, as well as procedures for recombinant production and purification of antibodies are well-known in the art. A description of such techniques, including relevant references, is given e.g. in WO 2006/082515.

It is known that several mechanisms are involved in the therapeutic efficacy of antibodies against growth factor receptors such as EGFR. These include blocking of ligand (e.g., EGF, TGF-α etc.) binding to their receptor and subsequent activation of signaling pathways, antibody dependent cell-mediated cytotoxicity (ADCC), complement-dependent cytotoxicity (CDC) and the induction of growth arrest, apoptosis or terminal differentiation.

The therapeutic efficacy of the humanized anti-EGFR IgG1 antibody useful for the present invention can be enhanced by producing it in host cells that further express a polynucleotide encoding a polypeptide having β(1,4)-N-acteylglucosaminyltransferase (GnTIII) activity, as described in WO 2006/082515, which results in antibodies having a reduced proportion of fucosylated oligosaccharides in the Fc region (termed “partially fucosylated” antibodies). In a preferred aspect, the polypeptide having GnTIII activity is a fusion polypeptide comprising the catalytic domain of GnTIII and the Golgi localization domain of a heterologous Golgi resident polypeptide, such as the Golgi localization domain of mannosidase II, mannosidase I, β(1,2)-N-acteylglucosaminyltransferase I (GnTI), β(1,2)-N-acteylglucosaminyltransferase II (GnTII) or α1-6 core fucosyltransferase, preferably mannosidase II or GnTI. Methods for generating such fusion polypeptides and using them to produce antibodies with increased effector functions are disclosed in U.S. Provisional Patent Appl. No. 60/495,142 and U.S. Patent Appl. Publ. No. 2004/0241817 A1, the entire contents of each of which are expressly incorporated herein by reference.

The partially fucosylated humanized anti-EGFR IgG1 antibody exhibits increased Fc receptor binding affinity and/or increased effector function as a result of the oligosaccharide modification. Preferably, the increased Fc receptor binding affinity is increased binding to a Fcγ activating receptor, such as the FcγRIIIa receptor. The increased effector function is preferably an increase in one or more of the following: increased Fc-mediated cellular cytotoxicity (including increased antibody-dependent cell-mediated cytotoxicity (ADCC)), increased antibody-dependent cellular phagocytosis (ADCP), increased cytokine secretion, increased immune-complex-mediated antigen uptake by antigen-presenting cells, increased binding to NK cells, increased binding to macrophages, increased binding to polymorphonuclear cells (PMNs), increased binding to monocytes, increased crosslinking of target-bound antibodies, increased direct signaling inducing apoptosis, increased dendritic cell maturation, and increased T cell priming.

Partially fucosylated antibodies can be produced in a host cell expressing a polynucleotide encoding the antibody and a polynucleotide encoding a polypeptide with GnTIII activity, or a vector comprising such polynucleotides. Production of the humanized anti-EGFR IgG1 antibody in said host cell comprises the following steps (a) culturing a host cell engineered to express at least one nucleic acid encoding a polypeptide having GnTIII activity under conditions which permit the production of the antibody, wherein said polypeptide having GnTIII activity is expressed in an amount sufficient to modify the oligosaccharides in the Fc region of said antibody produced by said host cell; and (b) isolating said antibody.

A variety of host cells and expression vector systems can be used for the production of antibodies and are well known in the art. Suitable host cells for expressing the humanized EGFR IgG1 antibody useful for the invention include cultured cells, e.g. cultured mammalian cells such as CHO cells, HEK293-EBNA cells, BHK cells, NSO cells, SP2/0 cells, YO myeloma cells, P3X63 mouse myeloma cells, PER cells, PER.C6 cells or hybridoma cells, E. coli cells, yeast cells, insect cells, and plant cells, to name only a few, but also cells comprised within a transgenic animal, transgenic plant or cultured plant or animal tissue. Detailed information about the production of the humanized anti-EGFR IgG1 antibody can be found in WO 2006/082515 and the references cited therein.

The present invention provides a humanized anti-EGFR IgG1 antibody, as described hereinbefore, and irinotecan for combined use in treating cancer. The humanized anti-EGFR IgG1 antibody and irinotecan may be administered together or separately, simultaneously or sequentially, in the same or in different formulations, by the same or different routes, and with or without additional therapeutic agents or treatments, such as other anti-cancer agents or radiation therapy.

The invention also encompasses a pharmaceutical composition, in particular for use in treating cancer, which comprises a humanized anti-EGFR IgG1 antibody, as described hereinbefore, and irinotecan as active ingredients, a pharmaceutically acceptable carrier and optionally one or more other therapeutically active ingredients or adjuvants. Other therapeutic agents may include cytotoxic, chemotherapeutic or anti-cancer agents, or agents which enhance the effects of such agents.

The data presented in the Examples herein below demonstrate that co-administration of irinotecan with a humanized anti-EGFR IgG1 antibody is effective for treatment of advanced cancers, such as Non Small Cell Lung Cancer (NSCLC). Accordingly, the present invention provides a method for the treatment of cancer, characterized in that a therapeutically effective amount of a combination of a humanized anti-EGFR IgG1 antibody, as described hereinbefore, and irinotecan is administered to a subject in need of such treatment. A therapeutically effective amount of a combination of a humanized anti-EGFR IgG1 antibody and irinotecan (referred to as “active agents” hereinbelow) may be a therapeutically effective amount of each of the active agents. Alternatively, in order to reduce the side effects caused by the treatment of cancer, a therapeutically effective amount of a combination of a humanized anti-EGFR IgG1 antibody and irinotecan may be amounts of the two active agents that are effective to produce an additive, or a superadditive or synergistic antitumor effect, and that in combination are effective at inhibiting the growth of the tumor, but which may be sub-therapeutic amounts of one or both of the active agents if they were used alone. Preferably, in the method for the treatment of cancer according to the invention, the humanized anti-EGFR IgG1 antibody and irinotecan are intended for administration to the patient together or separately, simultaneously or sequentially, in the same or in different formulations, by the same or different routes, and with or without additional agents or treatments, such as other anti-cancer drugs or radiation therapy.

The present invention further provides a method for manufacturing a medicament for the treatment of cancer, characterized in that a therapeutically effective amount of a combination of a humanized anti-EGFR IgG1 antibody, as described hereinbefore, and irinotecan is used and the humanized anti-EGFR IgG1 antibody and irinotecan are intended for administration to the patient together or separately, simultaneously or sequentially, in the same or in different formulations, by the same or different routes, and with or without additional agents or treatments. As described above, a therapeutically effective amount of a combination of a humanized anti-EGFR IgG1 antibody and irinotecan may be a therapeutically effective amount of each of the active agents, or amounts of the two active agents that are effective to produce an additive, or a superadditive or synergistic antitumor effect, and that in combination are effective at inhibiting the growth of the tumor, but which may be sub-therapeutic amounts of one or both of the active agents if they were used alone.

The present invention further provides a kit, useful for the treatment of cancer, comprising a single container comprising both the humanized anti-EGFR IgG1 antibody, as described hereinbefore, and irinotecan. The present invention further provides a kit comprising a first container comprising the humanized anti-EGFR IgG1 antibody, as described hereinbefore, and a second container comprising irinotecan. In a preferred aspect, the kit containers may further include a pharmaceutically acceptable carrier. The kit may further include a sterile diluent, which is preferably stored in a separate additional container. The kit may further include a package insert comprising printed instructions directing the use of the combined treatment as a method for treating cancer.

The present invention is intended for the treatment of cancer. Accordingly the subject in need is a human, horse, swine, bovine, mouse, rat, dog, cat, bird or other warm-blooded animal, preferably a human, in need of treatment of cancer or a precancerous condition or lesion. The cancer is preferably any cancer treatable, either partially or completely, by administration of a combination of a humanized anti-EGFR IgG1 antibody, as described hereinbefore, and irinotecan i.e. a disorder that relates to EGFR expression, in particular, a cell proliferation disorder wherein EGFR is expressed, and more particularly, wherein EGFR is abnormally expressed (e.g. overexpressed). The cancer may be, for example, lung cancer, non small cell lung cancer (NSCLC), bronchioloalveolar carcinoma, bone cancer, pancreatic cancer, skin cancer, cancer of the head or neck, squameous cell carcinoma, cutaneous or intraocular melanoma, uterine cancer, ovarian cancer, colorectal cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, including refractory versions of any of the above cancers, or a combination of one or more of the above cancers. Also included are cancer metastases. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions. Preferably, the cancer is lung cancer or colorectal cancer, and most preferably non-small cell lung cancer (NSCLC).

In some aspects of this invention, the humanized anti-EGFR IgG1 antibody, as described hereinbefore, and irinotecan may be administered in combination with one or more anti-cancer agents. Preferably, said anti-cancer agents may be selected from the groups of microtubule disruptors (e.g. vinca alkaloids such as vinblastine or vincristine, taxanes such as docetaxel or paclitaxel, epothilones such as ixabepilone), antimetabolites (e.g. anti-folates such as methotrexate or aminopterin, anti-purines such as fludarabine, 6-mercaptopurine or 6-thioguanine, anti-pyrimidines such as 5-fluorouracil, capecitabine or gemcitabine, hydroxyurea), topoisomerase inhibitors (e.g. camptothecin, topotecan, or podophyllotoxins such as etoposide), DNA intercalators (e.g. doxorubicin, daunorubicin, actinomycin, bleomycin), alkylating agents (e.g. cyclophosphamide, chlorambucil, nitrosureas such as carmustine or nimustine, streptozocin, busulfan, cisplatin, oxaliplatin, triethylenemelamine, dacarbazine), hormonal therapies (e.g. glucocorticoids, aromatase inhibitors such as tamoxifene, antiandrogens such as flutamide, gonadotropin-releasing hormone (GnRH) analogs such as leuprolide), antibiotics, kinase inhibitors (e.g. erlotinib, gefitinib, imatinib), receptor antagonists, enzyme inhibitors (e.g. cyclin-dependent kinase (CDK) inhibitors), amino acid-depleting enzymes (e.g. asparaginase), leucovorin, retinoids, activators of tumor cell apoptosis, and antiangiogenic agents.

The humanized anti-EGFR IgG1 antibody useful for the invention may also be conjugated to a cytotoxic agent such as a chemotherapeutic agent, a toxin (e.g. an enzymatically active toxin of bacterial, fungal, plant or animal origin, or fragments thereof), a radioactive isotope, or to a prodrug of a cytotoxic agent.

The humanized anti-EGFR IgG1 antibody and irinotecan as used in the invention, or the pharmaceutical composition according to the invention can be administered in any effective manner known in the art, such as by oral, topical, intravenous, intraperitoneal, intralymphatic, intramuscular, intra-articular, subcutaneous, intranasal, intra-ocular, vaginal, rectal, or intradermal routes, or by injection directly into the tumor. The choice of a route of administration depends upon the type of cancer being treated and the medical judgement of the prescribing physician as based, e.g., on the results of published clinical studies. The humanized anti-EGFR IgG1 antibody and irinotecan can be administered by the same or by different routes. Preferably, the humanized anti-EGFR IgG1 antibody as used in the invention is intended for parenteral administration and irinotecan as used in the present invention is intended for parenteral or oral administration. Preferably, the pharmaceutical composition according to the invention, or the humanized anti-EGFR IgG1 antibody and irinotecan as used in the invention if administered by the same route, are administered parenterally, most preferably intravenously. The humanized anti-EGFR IgG1 antibody and irinotecan as used in the invention or the pharmaceutical composition according to the invention can be administered by controlled release means and/or delivery devices.

According to the present invention, the combination of a humanized anti-EGFR IgG1 antibody, as described hereinbefore, and irinotecan should be administered in a therapeutically effective amount, meaning that each of the active agents is given in a therapeutically effective dose, or that the amounts of the two active agents are effective to produce an additive, or a superadditive or synergistic antitumor effect, so that in combination they are effective at inhibiting the growth of the tumor, although they would be sub-therapeutic amounts if the active agents were used alone.

Dosage levels for the compounds of the combination of this invention will be approximately as described below, or as described in the art for these compounds. The most effective mode of administration and dosage regimen for the humanized anti-EGFR IgG1 antibody and irinotecan as used in the invention, or the pharmaceutical compositions according to this invention, depends on a variety of factors, including the severity and course of the disease, the patient's general health, age, body weight, sex, diet and response to treatment, the time and route of administration, the rate of excretion, combinations with other drugs, and the judgment of the treating physician. Accordingly, the dosages of the humanized anti-EGFR IgG1 antibody and irinotecan, or the compositions, should be titrated to the individual patient. Nevertheless, a therapeutically effective dose of the humanized anti-EGFR IgG1 antibody as used in this invention will generally be in the range of from about 0.01 to about 2000 mg/kg. Typically, the therapeutically effective amount of the antibody administered parenterally per dose will be in the range of from about 1 to 25 mg/kg of patient body weight per day. In one aspect, the effective dose is in the range of from about 1.0 mg/kg to about 25.0 mg/kg. In a more specific aspect, the dose is in the range of from about 1.5 mg/kg to about 15 mg/kg. In other aspects, the dose is in the range of from about 1.5 mg/kg to about 4.5 mg/kg, or from about 4.5 mg/kg to about 15 mg/kg. The dose of the present invention may also be any dose within these ranges, including, but not limited to, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 14.5 mg/kg, or 15.0 mg/kg. A therapeutically effective dose of irinotecan as used in the present invention will generally be in the range from about 0.1 to about 2000 mg/kg. Typically, the therapeutically effective amount of irinotecan administered parenterally per dose will be in the range of from about 1 to 25 mg/kg of patient body weight per day, or in the range of from about 10 to about 1000 mg/m². In a more specific aspect, the effective dose of irinotecan is in the range of from about 1 to about 10 mg/kg, or in the range of from about 20 to about 500 mg/m². The dose of the present invention may also be any dose within these ranges, including but not limited to 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, or including but not limited to 25 mg/m², 50 mg/m², 75 mg/m², 100 mg/m², 125 mg/m², 150 mg/m², 175 mg/m², 200 mg/m², 225 mg/m², 250 mg/m², 275 mg/m², 300 mg/m², 325 mg/m², 350 mg/m², 375 mg/m², 400 mg/m², 425 mg/m², 450 mg/m², 475 mg/m², 500 mg/m².

As noted above, however, these suggested amounts of humanized anti-EGFR IgG1 antibody and of irinotecan are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the antagonist is administered as close to the first sign, diagnosis, appearance, or occurrence of the disease or disorder as possible or during remissions of the disease or disorder.

In the case of anti-EGFR antibodies used to treat tumors, optimum therapeutic results have generally been achieved with a dose that is sufficient to completely saturate the EGF receptors on the target cells. The dose necessary to achieve saturation will depend on the number of EGF receptors expressed per tumor cell (which can vary significantly between different tumor types). Serum concentrations as low as 30 nM have been effective in treating some tumors, while concentrations above 100 nM may be necessary to achieve optimum therapeutic effect with other tumors. The dose necessary to achieve saturation for a given tumor can be readily determined in vitro by radioimmunoassay or immunoprecipitation.

The dosages of the present invention may, in some cases, be determined by the use of predictive biomarkers. Predictive biomarkers are molecular markers that are used to determine (i.e., observe and/or quanitate) a pattern of expression and/or activation of tumor-related genes or proteins, or cellular components of a tumor-related signalling pathway. Elucidating the biological effects of targeted therapies in tumor tissue and correlating these effects with clinical response helps identify the predominant growth and survival pathways operative in tumors, thereby establishing a profile of likely responders and conversely providing a rationale for designing strategies to overcome resistance. For example, biomarkers for anti-EGFR therapy may comprise molecules that are in the EGFR downstream signalling pathway that leads to a cell proliferation disorder including, but not limited to, Akt, RAS, RAF, MAPK, ERK1, ERK2, PKC, STAT3, STAT5 (Mitchell, Nat Biotech 22, 363-364 (2004); Becker, Nat Biotech 22; 15-18 (2004); Tsao and Herbst, Signal 4, 4-9 (2003)). Biomarkers for anti-EGFR therapy may also comprise growth factor receptors such as EGFR, ErbB-2 (HER2/neu), and ErbB-3 (HER3), and may be positive or negative predictors of patient response to anti-EGFR therapy. For example, the growth factor receptor ErbB-3 (HER3) was determined to be a negative predictive biomarker for the anti-EGFR antibody ABX-EGF (U.S. Patent Appl. Pub. No. 2004/0132097 A1).

Predictive biomarkers may be measured by assays that are well known in the art including, but not limited to detection and/or quantification of RNA by real-time reverse transcription PCR or microarray-based transcriptional profiling, detection and/or quantification of protein by immunohistochemistry, flow cytometry, immunofluorescence, capture-and-detection assays, Western blot, ELISA, reversed phase assays, and/or assays set forth in U.S. Patent Appl. Pub. No. 2004/0132097 A1, the entire contents of which are herein incorporated by reference. Predictive biomarkers for anti-EGFR therapy can be identified according to the techniques set forth in U.S. Patent Appl. Pub. No. 2003/0190689A1, the entire contents of which are hereby incorporated by reference.

In one aspect, the present invention provides a method for treating an EGFR-related disorder comprising predicting a response to anti-EGFR therapy in a human subject in need of treatment by assaying a sample from the human subject prior to therapy with one or a plurality of reagents that detect expression and/or activation of predictive biomarkers for an EGFR-related disorder such as cancer; determining a pattern of expression and/or activation of one or more of the predictive biomarkers, wherein the pattern predicts the human subject's response to the anti-EGFR therapy; and administering to a human subject who is predicted to respond positively to anti-EGFR treatment a therapeutically effective amount of a composition comprising the humanized anti-EGFR IgG1 antibody. As used herein, “a human subject who is predicted to respond positively to anti-EGFR treatment” is one for whom anti-EGFR will have a measurable effect on the EGFR-related disorder (e.g., tumor regression/shrinkage) and for whom the benefits of anti-EGFR therapy are not outweighed by adverse effects (e.g. toxicity). As used herein, a sample means any biological sample from an organism, particularly a human, comprising one or more cells, including single cells of any origin, tissue or biopsy samples which has been removed from organs such as breast, lung, gastrointestinal tract, skin, cervix, ovary, prostate, kidney, brain, head and neck, or any other organ or tissue of the body, and other body samples including, but not limited to, smears, sputum, secretions, cerebrospinal fluid, bile, blood, lymph fluid, urine and feces.

For purposes of the present invention, “co-administration of”, “co-administering”, “administering a combination” and “combining” of a humanized anti-EGFR IgG1 antibody and irinotecan refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. Irinotecan can be administered prior to, at the same time as, or subsequent to the administration of the humanized anti-EGFR IgG1 antibody, or in some combination thereof. Where the humanized anti-EGFR IgG1 antibody is administered to the patient at repeated intervals, e.g., during a standard course of treatment, irinotecan can be administered prior to, at the same time as, or subsequent to, each administration of the humanized anti-EGFR IgG1 antibody or some combination thereof, or at different intervals in relation to the humanized anti-EGFR IgG1 antibody treatment, or in a single dose prior to, at any time during, or subsequent to the course of treatment with the humanized anti-EGFR IgG1 antibody.

The humanized anti-EGFR IgG1 antibody will typically be administered to the patient in a dose regimen that provides for the most effective treatment of the cancer (from both efficacy and safety perspectives) for which the patient is being treated, as known in the art, and as disclosed, e.g. in WO 2006/082515.

As discussed above, the amount of the humanized anti-EGFR IgG1 antibody administered and the timing of the antibody administration will depend on the type (species, gender, age, weight, etc.) and condition of the patient being treated, the severity of the disease or condition being treated, and on the route of administration. For example, the humanized anti-EGFR IgG1 antibody can be administered to a patient in doses ranging from 0.1 to 100 mg/kg of body weight per day or per week in single or divided doses, or by continuous infusion. In some instances, dosage levels below the lower limit of the aforesaid range may be adequate, while in other cases still larger doses may be employed without causing any harmful side effect, provided that such larger doses are first divided into several small doses for administration throughout the day. The same holds true for the amount of irinotecan administered and the timing of the irinotecan administration.

The humanized anti-EGFR IgG1 antibody and irinotecan as used according to the invention can be administered either separately or together by the same or different routes, and in a wide variety of different dosage forms.

Both the humanized anti-EGFR IgG1 antibody and irinotecan as used in the invention, as well as the pharmaceutical compositions according to the invention, may be in a variety of dosage forms which include, but are not limited to, liquid solutions or suspensions, emulsions, tablets, pills, dragées, powders, ointments, creams, suppositories or implants. The humanized anti-EGFR IgG1 antibody and/or irinotecan as used in the invention or the compositions according to the invention may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Mack Pub. Co. (1980).The preferred dosage form depends upon the mode of administration and the therapeutic application. Typically, the humanized anti-EGFR IgG1 antibody and irinotecan as used in the present invention, or the pharmaceutical compositions according to the invention, will be administered in injectable or infusible solutions. Injectable or infusible preparations must be sterile, which is readily accomplished by filtration through sterile filtration membranes.

Sustained-release preparations may be prepared, such as membrane-controlled sustained release systems, or polymer-based matrix systems. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The humanized anti-EGFR IgG1 antibody and irinotecan as used in the invention or pharmaceutical compositions according to the invention may be provided as bulk or conveniently presented in unit dosage forms, prepared by any of the methods well known in the art of pharmacy. Such unit dosage forms may e.g. be suitable for oral administration (capsules, cachets, tablets, etc.) and each contain a predetermined amount of the active ingredient(s).

The humanized anti-EGFR IgG1 antibody and irinotecan as used in the invention, as well as the pharmaceutical composition according to the invention, will be formulated, dosed, and administered in a fashion consistent with good medical practice.

The optimal formulation of the humanized anti-EGFR IgG1 antibody and irinotecan as used for the invention, as well as the pharmaceutical composition according to the invention will depend on the particular disease or disorder being treated, the particular mammal being treated, the clinical condition of the individual patient, the cause of the disease or disorder, the site of delivery of the agent, the route of administration (e.g. parenteral, oral, topical, rectal), the scheduling of the administration, and other factors known to medical practitioners.

All formulations should be selected so as to avoid denaturation and/or degradation and loss of biological activity of the humanized anti-EGFR IgG1 antibody and/or to preserve the integrity and biological activity of irinotecan.

In practice, the humanized anti-EGFR IgG1 antibody and/or irinotecan as used in the invention can be combined as the active ingredient in intimate admixture with a pharmaceutical carrier according to conventional pharmaceutical compounding techniques. The carrier may take a wide variety of forms depending on the type of preparation desired for administration, e.g. parenteral (including intravenous). The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In addition to the carrier ingredients, the pharmaceutical formulations may also contain, as appropriate, other ingredients such as buffers, diluents, solvents, stabilizers, antioxidants, agents to render the formulation isotonic, flavoring agents, binders, surface-active agents, thickeners, lubricants, preservatives, wetting agents, emulsifying agents, dispersing agents, agents to disintegrate tablets and the like. The formulations may be prepared by any of the methods of pharmacy.

Pharmaceutical formulations containing the humanized anti-EGFR IgG1 antibody and/or irinotecan as used in the present invention or the pharmaceutical compositions according to the invention, which are suitable for injection include sterile aqueous solutions or dispersions. Furthermore, the active agents and compositions can be in the form of sterile powders for the extemporaneous preparation of such sterile injectable solutions or dispersions. In all cases, the final injectable form must be sterile and must be effectively fluid for easy syringability. The formulations must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

For parenteral administration of either or both of the active agents, solutions in sesame or peanut oil or in aqueous propylene glycol may be employed, as well as sterile aqueous solutions comprising the active agent or a corresponding water-soluble salt thereof. Such sterile aqueous solutions are preferably suitably buffered, e.g. with histidine, acetate or phosphate buffers, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Therapeutic formulations containing the humanized anti-EGFR IgG1 antibody and/or irinotecan are prepared by mixing the active ingredient having the desired degree of purity with optional pharmaceutically acceptable carriers, solvents, excipients or stabilizers (Remington's Pharmaceutical Sciences, 16th edition, Mack Pub. Co. (1980)). They may be stored in the form of lyophilized formulations or aqueous solutions. Acceptable carriers, solvents, excipients, or stabilizers are non-toxic to recipients at the dosages and concentrations employed, and include e.g. buffers such as phosphate, citrate, histidine, acetate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyoxyethylen-sorbitan fatty acid esters (Tween™) or polyoxyethylene-polyoxypropylene copolymers (Pluronic™).

Lyophilized formulations adapted for subcutaneous administration are described in WO 97/04801. Such lyophilized formulations may be reconstituted with a suitable diluent to a high protein concentration and the reconstituted formulation may be administered subcutaneously to the mammal to be treated herein.

Methods of preparing pharmaceutical compositions comprising antibodies or antigen binding fragments thereof are known in the art, and are described, e.g. WO 2006/082515. Methods of preparing pharmaceutical compositions comprising irinotecan are also known in the art (e.g. Rothenberg et al., J Clin Oncol 11, 2194-2204 (1993). Methods of preparing pharmaceutical compositions comprising the humanized anti-EGFR IgG1 antibody and irinotecan will be apparent from the above-cited publications and from other known references, such as Remington's Pharmaceutical Sciences, 18th edition, Mack Pub. Co. (1990). The combination compositions may be prepared by any of the methods of pharmacy.

The examples below explain the invention in more detail. The following preparations and examples are given to enable those skilled in the art to more clearly understand and to practice the present invention. The present invention, however, is not limited in scope by the exemplified aspects, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

Terms are used herein as generally used in the art, unless otherwise defined as follows.

As used herein, the term “antibody” is intended to include whole antibody molecules, including monoclonal, polyclonal and multispecific (e.g., bispecific) antibodies, as well as antibody fragments having the Fc region and retaining binding specificity, and fusion proteins that include a region equivalent to the Fc region of an immunoglobulin and that retain binding specificity. Also encompassed are antibody fragments that retain binding specificity including, but not limited to, V_{H} fragments, V_{L} fragments, Fab fragments, F(ab′)₂ fragments, scFv fragments, Fv fragments, minibodies, diabodies, triabodies, and tetrabodies (see e.g. Hudson and Souriau, Nat Med 9, 129-134 (2003)). Also encompassed are genetically engineered, recombinant, humanized, primatized and chimeric antibodies, as well as antibodies from different species such as mouse or human.

As used herein, the terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a human light chain transgene, fused to an immortalized cell.

As used herein, the term “humanized” is used to refer to an antigen-binding molecule derived from a non-human antigen-binding molecule, for example, a murine antibody, that retains or substantially retains the antigen-binding properties of the parent molecule but which is less immunogenic in humans, e.g. chimeric antibodies. Reduction of immunogenicity may be achieved by various methods including (a) grafting the entire non-human variable domains onto human constant regions to generate chimeric antibodies, (b) grafting only the non-human CDRs onto human framework and constant regions with or without retention of critical framework residues (e.g., those that are important for retaining good antigen binding affinity or antibody functions), (c) grafting only the non-human specificity-determining regions (SDRs; the residues critical for the antibody-antigen interaction) onto human framework and constant regions, or (d) transplanting the entire non-human variable domains, but “cloaking” them with a human-like section by replacement of surface residues. Such methods are disclosed in Morrison et al., Proc Natl Acad Sci USA 81, 6851-6855 (1984); Morrison and Oi, Adv Immunol 44, 65-92 (1988); Verhoeyen et al., Science 239, 1534-1536 (1988); Padlan, Molec Immun 28, 489-498 (1991); Padlan, Molec Immun 31(3), 169-217 (1994), Kashmiri et al., Methods 36, 25-34 (2005), all of which are incorporated by reference in their entirety herein. There are generally three complementarity determining regions, or CDRs, (CDR1, CDR2 and CDR3) in each of the heavy and light chain variable domains of an antibody, which are flanked by four framework subregions (i.e. FR1, FR2, FR3, and FR4) in each of the heavy and light chain variable domains of an antibody: FR1-CDR1-FR2-CDR2-FR3-CDR3-FR4. A discussion of humanized antibodies can be found, inter alia, in U.S. Pat. No. 6,632,927, and in published U.S. Application No. 2003/0175269, both of which are incorporated herein by reference in their entirety.

Similarly, as used herein, the term “primatized” refers to an antibody derived from a non-primate antibody, e.g. a murine antibody, that retains or substantially retains the antigen-binding properties of the parent molecule, but which is less immunogenic in primates.

The “variable region” or “variable domain” (variable region of a light chain (V_{L}), variable region of a heavy chain (V_{H})) as used herein denotes each of the pair of light and heavy chains which is involved directly in binding the antibody to the antigen. The human light and heavy chain variable domains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies useful for the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues of the “complementarity determining regions” or “CDRs”. “Framework” or “FR” regions are those variable domain regions other than the hypervariable region residues as herein defined. Therefore, the variable regions of the light and heavy chains of an antibody comprise from N- to C-terminus the domains FR1, CDR1, FR2, CDR2, FR3, CDR3, and FR4. Notably, CDR3 of the heavy chain is the region which contributes most to antigen binding. CDR and FR regions can be determined according to the standard definition of Kabat et al., “Sequences of Proteins of Immunological Interest”, 5th Ed. Public Health Service, National Institutes of Health, Bethesda, Md. (1991)) and/or those residues from a “hypervariable loop”.

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen binding sites found within the variable region of both heavy and light chain polypeptides. This particular region has been described by Kabat et al., “Sequences of Proteins of Immunological Interest”, National Institutes of Health, Bethesda (1983) and by Chothia et al., J Mol Biol 196, 901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 4 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 4
CDR Definitions₁

| | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 5 is according to the numbering conventions set forth by Kabat et al. (see below).
²“AbM” refers to the CDRs as defined by Oxford Molecular's “AbM” antibody modeling software.

Kabat et al. also defined a numbering system for variable domain sequences that is applicable to any antibody. One of ordinary skill in the art can unambigously assign this system of “Kabat numbering” to any variable domain sequence, without reliance on any experimental data beyond the sequence itself. As used herein, “Kabat numbering” refers to the numbering system set forth by Kabat et al., “Sequences of Proteins of Immunological Interest”, National Institutes of Health, Bethesda (1983). Unless otherwise specified, references to the numbering of specific amino acid residue positions in an antigen binding molecule are according to the Kabat numbering system.

The “constant domains” are the parts of an antibody molecule other than the variable regions. They are not involved directly in binding the antibody to an antigen but are involved in the effector functions (e.g. ADCC, CDC). The constant domain of the antibodies useful for the invention is preferably of the IgG1 isotype. Human constant domains having these characteristics are described in detail by Kabat et al., “Sequences of Proteins of Immunological Interest”, National Institutes of Health, Bethesda (1991), and by Brüuggemann et al., J Exp Med 166, 1351-1361 (1987); Love et al., Methods Enzymol 178, 515-527 (1989). The constant domains useful in the invention provide complement binding and Fc receptor binding. ADCC and optionally CDC are provided by the combination of variable and constant domains.

As used herein, the term “Fc region” is intended to refer to a C-terminal region of an IgG heavy chain. Although the boundaries of the Fc region of an IgG heavy chain might vary slightly, the human IgG heavy chain Fc region is usually defined to stretch from the amino acid residue at position Cys 226 to the carboxyl-terminus.

As used herein, the term “region equivalent to the Fc region of an immunoglobulin” is intended to include naturally occurring allelic variants of the Fc region of an immunoglobulin, as well as variants having alterations which produce substitutions, additions, or deletions but which do not decrease substantially the ability of the immunoglobulin to mediate effector functions (such as antibody dependent cell-mediated cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see e.g. Bowie et al., Science 247, 1306-1310 (1990).

As used herein, “a polypeptide having GnTIII activity” refers to polypeptides that are able to catalyze the addition of a N-acetylglucosamine (GlcNAc) residue in β-1-4 linkage to the β-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-β-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependence in a given activity as compared to the GnTIII (i.e., the candidate polypeptide will exhibit greater activity or not more than about 25-fold less and, preferably, not more than about tenfold less activity, and most preferably, not more than about threefold less activity relative to the GnTIII).

As used herein, the term “Golgi localization domain” refers to the amino acid sequence of a Golgi-resident polypeptide which is responsible for anchoring the polypeptide in location within the Golgi complex. Generally, localization domains comprise amino terminal “tails” of an enzyme.

As used herein, the term “host cell” covers any kind of cellular system which can be engineered to generate the antibodies of the present invention. In one embodiment, the host cell is engineered to allow the production of an antibody with modified glycoforms. Preferably, the host cells have been engineered to express increased levels of one or more polypeptides having GnTIII activity. Host cells include cultured cells, e.g. cultured mammalian cells such as CHO cells, HEK293-EBNA cells, BHK cells, NSO cells, SP2/0 cells, YO myeloma cells, P3X63 mouse myeloma cells, PER cells, PER.C6 cells or hybridoma cells, E. coli cells, yeast cells, insect cells, and plant cells, to name only a few, but also cells comprised within a transgenic animal, transgenic plant or cultured plant or animal tissue.

As used herein, the term “effector function” refers to those biological activities attributable to the Fc region (a native sequence Fc region or amino acid sequence variant Fc region) of an antibody. Examples of antibody effector functions include, but are not limited to, Fc receptor binding affinity, antibody-dependent cell-mediated cytotoxicity (ADCC), antibody-dependent cellular phagocytosis (ADCP), cytokine secretion, immune complex-mediated antigen uptake by antigen-presenting cells, down-regulation of cell surface receptors, etc.

The terms “engineer”, “engineered”, “engineering”, “glycosylation engineering”, “glycoengineered”, as used herein, includes any manipulation of the glycosylation pattern of a naturally occurring or recombinant protein, polypeptide or a fragment thereof. Glycoengineering includes metabolic engineering of the glycosylation machinery of a cell, including genetic manipulations of the oligosaccharide synthesis pathways to achieve altered glycosylation of glycoproteins expressed in these cells. Furthermore, glycoengineering includes the effects of mutations and cell environment on glycosylation. In particular, glycoengineering can result in altered glycosyltransferase activity, such as altered glucosaminyltransferase and/or fucosyltransferase activity.

As used herein, the term “Fe-mediated cellular cytotoxicity” includes antibody-dependent cell-mediated (sometimes also termed “cellular”) cytotoxicity (ADCC) and cellular cytotoxicity mediated by a soluble Fc-fusion protein containing a human Fc-region. It is an immune mechanism leading to the lysis of “antibody-targeted cells” by “human immune effector cells”, wherein:

The “human immune effector cells” are a population of leukocytes that display Fc receptors on their surface through which they bind to the Fc-region of antibodies or of Fc-fusion proteins and perform effector functions. Such a population may include, but is not limited to, peripheral blood mononuclear cells (PBMC) and/or natural killer (NK) cells.

The “antibody-targeted cells” are cells bound by the antibodies or Fc-fusion proteins. The antibodies or Fc fusion-proteins bind to target cells via the protein part N-terminal to the Fc region.

As used herein, the term “increased Fc-mediated cellular cytotoxicity” is defined as either an increase in the number of “antibody-targeted cells” that are lysed in a given time, at a given concentration of antibody or of Fc-fusion protein, in the medium surrounding the target cells, by the mechanism of Fc-mediated cellular cytotoxicity defined above, and/or a reduction in the concentration of antibody, or of Fc-fusion protein, in the medium surrounding the target cells, required to achieve the lysis of a given number of “antibody-targeted cells”, in a given time, by the mechanism of Fc-mediated cellular cytotoxicity. The increase in Fc-mediated cellular cytotoxicity is relative to the cellular cytotoxicity mediated by the same antibody, or Fc-fusion protein, produced by the same type of host cells, using the same standard production, purification, formulation and storage methods, which are known to those skilled in the art, but that has not been produced by host cells engineered to express the glycosyltransferase GnTIII by the methods described herein.

By “antibody having increased antibody dependent cell-mediated cytotoxicity (ADCC)” is meant an antibody, as that term is defined herein, having increased ADCC as determined by any suitable method known to those of ordinary skill in the art. One accepted in vitro ADCC assay is as follows:

1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:

i) the PBMCs are isolated using standard density centrifugation procedures and are suspended at 5×10⁶ cells/ml in RPMI cell culture medium;

ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;

iii) 100 microliters of the final target cell suspension, prepared as described above, are transferred to each well of a 96-well microtiter plate;

iv) the antibody is serially diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;

v) for the maximum release (MR) controls, 3 additional wells in the plate containing the labeled target cells, receive 50 microliters of a 2% (v/v) aqueous solution of non-ionic detergent (Nonidet, Sigma, St. Louis), instead of the antibody solution (point iv above);

vi) for the spontaneous release (SR) controls, 3 additional wells in the plate containing the labeled target cells, receive 50 microliters of RPMI cell culture medium instead of the antibody solution (point iv above);

vii) the 96-well microtiter plate is then centrifuged at 50×g for 1 minute and incubated for 1 hour at 4° C.;

viii) 50 microliters of the PBMC suspension (point i above) are added to each well to yield an effector:target cell ratio of 25:1 and the plates are placed in an incubator under 5% CO₂ atmosphere at 37° C. for 4 hours;

ix) the cell-free supernatant from each well is harvested and the experimentally released radioactivity (ER) is quantified using a gamma counter;

x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);

4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. The increase in ADCC is relative to the ADCC, measured with the above assay, mediated by the same antibody, produced by the same type of host cells, using the same standard production, purification, formulation and storage methods, which are known to those skilled in the art, but that has not been produced by host cells engineered to overexpress GnTIII.

As used herein, the term “variant” (or “analog”) refers to a polypeptide differing from a specifically recited polypeptide of the invention by amino acid insertions, deletions, and substitutions, created using, e.g., recombinant DNA techniques. Variants of the antibodies of the present invention include chimeric, primatized or humanized antibodies wherein one or several of the amino acid residues are modified by substitution, addition and/or deletion in such manner that does not substantially affect antigen (e.g., EGFR) binding affinity. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to a reference polypeptide can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp App Biosci 6, 237-245 (1990).

As used herein, the term “EGFR” refers to the human epidermal growth factor receptor (also known as HER-1 or ErbB-1) (Ulrich et al., Nature 309, 418-425 (1984); SwissProt Accession #P00533; secondary accession numbers: O00688, O00732, P06268, Q14225, Q68GS5, Q92795, Q9BZS2, Q9GZX1, Q9H2C9, Q9H3C9, Q9UMD7, Q9UMD8, Q9UMG5), as well as naturally-occurring isoforms and variants thereof. Such isoforms and variants include but are not limited to the EGFRvIII variant, alternative splicing products (e.g., as identified by SwissProt Accession numbers P00533-1, P00533-2, P00533-3, P00533-4), variants GLN-98, ARG-266, Lys-521, ILE-674, GLY-962, and PRO-988 (Livingston et al., NIEHS-SNPs, environmental genome project, NIEHS ES15478, Department of Genome Sciences, Seattle, Wash. (2004)), and others identified by the following accession numbers: NM_{—}005228.3, NM_{—}201282.1, NM_{—}201283.1, NM_{—}201284.1 (REFSEQ mRNAs); AF125253.1, AF277897.1, AF288738.1, AI217671.1, AK127817.1, AL598260.1, AU137334.1, AW163038.1, AW295229.1, BC057802.1, CB160831.1, K03193.1, U48722.1, U95089.1, X00588.1, X00663.1; H5448451, H5448453, H5448452 (MIPS assembly); DT.453606, DT.86855651, DT.95165593, DT.97822681, DT.95165600, DT.100752430, DT.91654361, DT.92034460, DT.92446349, DT.97784849, DT.101978019, DT.418647, DT.86842167, DT.91803457, DT.92446350, DT.95153003, DT.95254161, DT.97816654, DT.87014330, DT.87079224 (DOTS Assembly).

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, the term “ligand” refers to a polypeptide which binds to and/or activates a receptor, such as EGFR. The term includes membrane-bound precursor forms of the ligand, as well as proteolytically processed soluble forms of the ligand.

As used herein, the term “ligand activation of EGFR” refers to signal transduction (e.g., that caused by an intracellular kinase domain of EGFR phosphorylating tyrosine residues in the receptor itself or a substrate polypeptide) mediated by EGFR ligand binding.

As used herein, the term “disease or disorder characterized by abnormal activation or production of EGFR or an EGFR ligand or disorder related to EGFR expression”, refers to a condition, which may or may not involve malignancy or cancer, where abnormal activation and/or production of EGFR, and/or an EGFR ligand is occurring in cells or tissues of a subject having, or predisposed to, the disease or disorder.

As used herein, the terms “overexpress”, “overexpressed”, and “overexpressing”, as used in connection with cells expressing EGFR, refer to cells which have measurably higher levels of EGFR on the surface thereof compared to a normal cell of the same tissue type. Such overexpression may be caused by gene amplification or by increased transcription or translation. EGFR expression (and, hence, overexpression) may be determined in a diagnostic or prognostic assay by evaluating levels of EGFR present on the surface of a cell or in a cell lysate by techniques that are known in the art: e.g., via an immunohistochemistry assay, immunofluorescence assay, immunoenzyme assay, ELISA, flow cytometry, radioimmunoassay, Western blot, ligand binding, kinase activity, etc. (see generally, Cell Biology: A Laboratory Handbook, Celis, J., ed., Academic Press (2nd ed., 1998); Current Protocols in Protein Science, Coligan, J. E. et al., eds., John Wiley & Sons (1995-2003); see also Sumitomo et al., Clin Cancer Res 10, 794-801 (2004), the entire contents of which are herein incorporated by reference). Alternatively, or additionally, one may measure levels of EGFR-encoding nucleic acid molecules in the cell, e.g., via fluorescent in situ hybridization, Southern blotting, or PCR techniques. The levels of EGFR in normal cells are compared to the levels of cells affected by a cell proliferation disorder (e.g., cancer) to determine if EGFR is overexpressed.

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expression of a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinase expression and/or activation; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The patient may be a human or an animal. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in a human or animal, prevent the progression of a cancer, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a human or animal, is nevertheless deemed an overall beneficial course of action.

The term “therapeutically effective or therapeutic agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

As used herein, the term “irinotecan” includes irinotecan as well as pharmaceutically acceptable salts thereof (e.g. irinotecan hydrochloride trihydrate). Specific, particularly suitable polymorphic forms are also included.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

All patents, published patent applications and other references disclosed herein are hereby expressly incorporated herein by reference in their entirety.

Those skilled in the art will recognize, or be able to ascertain, using no more than routine experimentation, many equivalents to specific aspects of the invention described specifically herein. Such equivalents are intended to be encompassed in the scope of the following claims.

### DESCRIPTION OF THE FIGURES

FIG. 1. Kaplan-Meier curves representing survival of SCID beige mice bearing A549 lung adenocarcinoma xenografts, treated with vehicle (solid line), 25 mg/kg partially fucosylated GlycArt-mAb and 20 mg/kg CPT-11/irinotecan (dashed line) or 25 mg/kg cetuximab (Erbitux™) and 20 mg/kg CPT-11/irinotecan (dotted line).

### EXAMPLES

### Example 1

### Survival of Mice Bearing Lung Adenocarcinoma Xenografts, Treated with Combinations of Anti-EGFR Antibodies and Irinotecan

### Test Agents

GlycArt-mAb is manufactured by techniques generally known from the production of recombinant proteins. Generation of cell lines for the production of humanized anti-EGFR IgG1 antibodies with altered glycosylation pattern, identification of transfectants or transformants that express the antibodies having a modified glycosylation pattern and generation of humanized anti-EGFR IgG1 antibodies having increased effector function including antibody-dependent cell-mediated cytotoxicity (ADCC) are described in detail in WO 2006/082515 and WO 2008/017963. Briefly, genetically engineered Chinese hamster ovary cell lines (CHO) are expanded in cell culture from a master cell bank. The antibodies are purified from the conditioned cell culture medium using protein A affinity chromatography on a MabSelect SuRe™ column (GE), followed by cation exchange chromatography on a Capto S™ column (GE) and a final anion exchange chromatographic step on a Capto Q™ column (GE). Viruses are removed by nanofiltration using a Viresolve® Pro membrane (Millipore) and the antibodies are concentrated and transferred into the desired buffer by diafiltration.

For the manufacture of partially fucosylated GlycArt-mAb, CHO cell lines overexpressing β(1,4)-N-acetylglucosaminyltransferase III (GnTIII) are used, as described in U.S. Pat. No. 7,517,670, and specifically described in WO 2006/082515 and WO 2008/017963.

Partially fucosylated GlycArt-mAb was provided as stock solution (c=11.3 mg/ml), in a buffer containing histidine, trehalose and polysorbate 20. The antibody stock solution was appropriately diluted in PBS prior to injection.

The anti-EGFR antibody cetuximab (Erbitux®) was purchased as clinical formulation (5 mg/ml) from Merck Pharma GmbH, Darmstadt, Germany. The antibody concentration was adjusted by dilution of the reconstituted stock solution prior to injection.

Irinotecan/CPT-11 (Campto®) was purchased as clinical formulation (20 mg/ml) from Pfizer Pharma GmbH, Karlsruhe, Germany. The antibody concentration was adjusted by dilution of the reconstituted stock solution prior to injection.

### Cell Lines and Culture Conditions

A549 human Non Small Cell Lung Cancer (NSCLC) cells were obtained from ATCC. The tumor cell line was routinely cultured in RPMI medium (PAA Laboratories, Austria) supplemented with 10% fetal bovine serum (PAA Laboratories, Austria) and 2 mM L-glutamine, at 37° C. in a water-saturated atmosphere at 5% CO₂.

### Tumor Cell Injection

Passage 3 of A549 cells was used for in vivo injection. 2×10⁶ cells in PBS were injected intravenously (i.v.).

### Animals

Female SCID beige mice; age 7-8 weeks at arrival (purchased from Charles River, Sulzfeld, Germany) were maintained under specific pathogen-free conditions with daily cycles of 12 h light/12 h darkness according to committed guidelines (GV-Solas; Felasa; TierschG). The experimental study protocol was reviewed and approved by local government. After arrival animals were maintained in the quarantine part of the animal facility for one week to get accustomed to the new environment and for observation. Continuous health monitoring was carried out on regular basis. Diet food (Provimi Kliba 3337) and water (acidified pH 2.5-3) were provided ad libitum.

### Monitoring

The animals were controlled daily for clinical symptoms and detection of adverse effects. For monitoring throughout the experiment the body weight of the animals was documented.

### Treatment of Animals

Animal treatment started after animal randomisation seven days after tumor cell injection. The GlycArt-mAb or the anti EGFR antibody cetuximab (Erbitux®) were administered intraperitoneally (i.p.) every seven days on study day 7, 14, 21, 28, 35, 42, 49, 56, 63, 70, 77, 84, 91 and finally on day 98 at the indicated dosage of 25 mg/kg. The corresponding vehicle was administered on the same days. Irinotecan was given i.p. as single agent or in combination with anti-EGFR antibodies four times on study day 7, 10, 14 and 17 at a dosage of 20 mg/kg.

### Animal Survival Study In Vivo

The in vivo anti-tumor efficacy of the combination of GlycArt-mAb with irinotecan, compared to the combination of the commercially available anti-EGFR antibody cetuximab with irinotecan, and both anti-EGFR antibodies and irinotecan as single agents was analyzed in the A549 lung adenocarcinoma xenograft model. The primary parameter was survival. The data was statistically analyzed by log rank test.

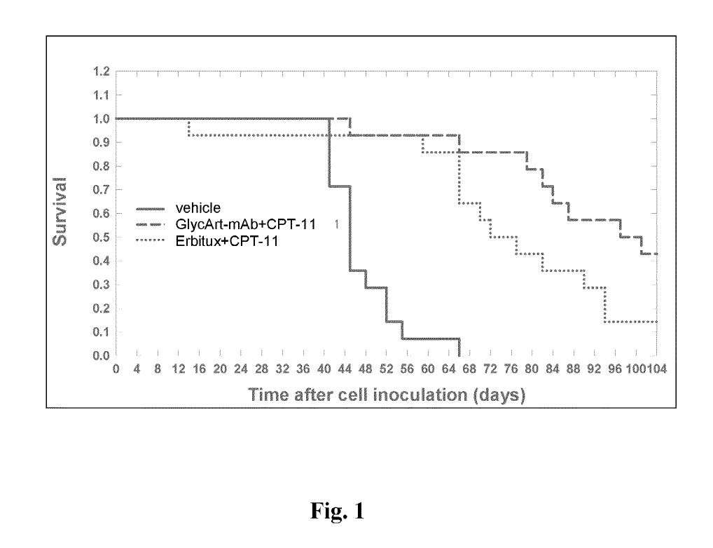

Treatment of mice bearing A549 xenografts (after i.v. injection) with both anti-EGFR antibodies GlycArt-mAb and cetuximab or irinotecan (CPT-11) as single agents significantly prolonged animal survival compared to vehicle control with p=0.0005, p=0.0031 and p=0.017, respectively. Furthermore, both combination treatments of anti-EGFR antibodies GlycArt-mAb or cetuximab with irinotecan improved animal median and long term survival compared to control (p<0.0001 and p=0.0003). For GlycArt-mAb, the combination with irinotecan was even superior compared to GlycArt-mAb or irinotecan as single agents (p=0.0116 and p=0.0001). Direct comparison of anti-EGFR antibody combinations with irinotecan highlighted the superiority for the combination of the GlycArt-mAb with irinotecan over cetuximab (Erbitux®) with irinotecan (p=0.246). Survival data are shown as Kaplan-Meier curves in FIG. 1.

1. A pharmaceutical composition comprising a humanized anti-EGFR IgG1 antibody, irinotecan, and a pharmaceutically acceptable carrier, wherein said humanized anti-EGFR IgG1 antibody comprises:
a) in the heavy chain variable domain a CDR1 with the amino acid sequence set forth in SEQ ID NO:1, a CDR2 with the amino acid sequence set forth in SEQ ID NO:16, and a CDR3 with the amino acid sequence set forth in SEQ ID NO:31; and
b) in the light chain variable domain a CDR1 with the amino acid sequence set forth in SEQ ID NO:33, a CDR2 with the amino acid sequence set forth in SEQ ID NO:34, and a CDR3 with the amino acid sequence set forth in SEQ ID NO:35,
and wherein the humanized anti-EGFR IgG1 antibody comprises an Fc-region comprising oligosaccharides.

2. The pharmaceutical composition of claim 1, wherein the oligosaccharides in the Fc-region of the humanized anti-EGFR IgG1 antibody are at least 20% non-fucosylated or bisected non-fucosylated oligosaccharides.

3. The pharmaceutical composition of claim 2, wherein the heavy chain of the humanized anti-EGFR IgG1 antibody comprises the amino acid sequence set forth in SEQ ID NO:38 and the light chain of the humanized anti-EGFR IgG1 comprises the amino acid sequence set forth in SEQ ID NO:39.

4. The pharmaceutical composition of claim 3, additionally comprising one or more other anti-cancer agents.

5. A kit for use in the treatment of cancer comprising irinotecan and a humanized anti-EGFR IgG1 antibody, where in the irinotecan and the humanized anti-EGFR IgG1 antibody are in the same or in separate containers, wherein said humanized anti-EGFR IgG1 antibody comprises
a) in the heavy chain variable domain a CDR1 with the amino acid sequence set forth in SEQ ID NO:1, a CDR2 with the amino acid sequence set forth in SEQ ID NO:16, and a CDR3 with the amino acid sequence set forth in SEQ ID NO:31; and
b) in the light chain variable domain a CDR1 with the amino acid sequence set forth in SEQ ID NO:33, a CDR2 with the amino acid sequence set forth in SEQ ID NO:34, and a CDR3 with the amino acid sequence set forth in SEQ ID NO:35,
and wherein the humanized anti-EGFR IgG1 antibody comprises an Fc-region comprising oligosaccharides.

6. The kit of claim 5, wherein oligosaccharides in the Fc-region of the humanized anti-EGFR IgG1 antibody are at least 20% non-fucosylated or bisected non-fucosylated oligosaccharides.

7. The kit of claim 6, wherein the heavy chain of the humanized anti-EGFR IgG1 antibody comprises the amino acid sequence set forth in SEQ ID NO:38 and the light chain of the humanized anti-EGFR IgG1 comprises the amino acid sequence set forth in SEQ ID NO:39

8. A method for the treatment of cancer, comprising administering to a subject in need of such treatment a therapeutically effective amount of a combination of a humanized anti-EGFR IgG1 antibody and irinotecan, wherein said humanized anti-EGFR IgG1 antibody comprises
a) in the heavy chain variable domain a CDR1 with the amino acid sequence set forth in SEQ ID NO:1, a CDR2 with the amino acid sequence set forth in SEQ ID NO:16, and a CDR3 with the amino acid sequence set forth in SEQ ID NO:31; and
b) in the light chain variable domain a CDR1 with the amino acid sequence set forth in SEQ ID NO:33, a CDR2 with the amino acid sequence set forth in SEQ ID NO:34, and a CDR3 with the amino acid sequence set forth in SEQ ID NO:35,
and wherein the humanized anti-EGFR IgG1 antibody comprises an Fc-region comprising oligosaccharides.

9. The method of claim 8, wherein the oligosaccharides in the Fc-region of the humanized anti-EGFR IgG1 antibody are at least 20% non-fucosylated or bisected non-fucosylated oligosaccharides.

10. The method of claim 9, wherein the heavy chain of the humanized anti-EGFR IgG1 antibody comprises the amino acid sequence set forth in SEQ ID NO:38 and the light chain of the humanized anti-EGFR IgG1 comprises the amino acid sequence set forth in SEQ ID NO:39.

11. The method of claim 8, wherein the humanized anti-EGFR IgG1 antibody and irinotecan are administered in the same formulation.

12. The method of claim 8, wherein the humanized anti-EGFR IgG1 antibody and irinotecan are administered in different formulations.

13. The method of claim 8, wherein the humanized anti-EGFR IgG1 antibody and irinotecan are administered by the same route.

14. The method of claim 8, which comprises administration of one or more other anti-cancer agents.


SEQUENCE LISTING (40 sequences)

SEQ ID NO: 1 - protein, 5 aa, Artificial Sequence
  Description of Artificial Sequence Synthetic peptide
DYKIH

SEQ ID NO: 2 - protein, 5 aa, Artificial Sequence
  Description of Artificial Sequence Synthetic peptide
DYAIS

SEQ ID NO: 3 - protein, 5 aa, Artificial Sequence
  Description of Artificial Sequence Synthetic peptide
DYYMH

SEQ ID NO: 4 - protein, 5 aa, Artificial Sequence
  Description of Artificial Sequence Synthetic peptide
DYKIS

SEQ ID NO: 5 - protein, 7 aa, Artificial Sequence
  Description of Artificial Sequence Synthetic peptide
GFTFTDY

SEQ ID NO: 6 - protein, 7 aa, Artificial Sequence
  Description of Artificial Sequence Synthetic peptide
GYTFTDY

SEQ ID NO: 7 - protein, 7 aa, Artificial Sequence
  Description of Artificial Sequence Synthetic peptide
GYSFTDY

SEQ ID NO: 8 - protein, 10 aa, Artificial Sequence
  Description of Artificial Sequence Synthetic peptide
GFTFTDYKIH

SEQ ID NO: 9 - protein, 10 aa, Artificial Sequence
  Description of Artificial Sequence Synthetic peptide
GFTFTDYAIS

SEQ ID NO: 10 - protein, 10 aa, Artificial Sequence
  Description of Artificial Sequence Synthetic peptide
GFTFTDYYMH

SEQ ID NO: 11 - protein, 10 aa, Artificial Sequence
  Description of Artificial Sequence Synthetic peptide
GYTFTDYYMH

SEQ ID NO: 12 - protein, 10 aa, Artificial Sequence
  Description of Artificial Sequence Synthetic peptide
GYSFTDYKIH

SEQ ID NO: 13 - protein, 10 aa, Artificial Sequence
  Description of Artificial Sequence Synthetic peptide
GFTFTDYKIS

SEQ ID NO: 14 - protein, 17 aa, Artificial Sequence
  Description of Artificial Sequence Synthetic peptide
YFNPNSGYSTYNEKFKS

SEQ ID NO: 15 - protein, 17 aa, Artificial Sequence
  Description of Artificial Sequence Synthetic peptide
GINPNSGYSTYAQKFQG

SEQ ID NO: 16 - protein, 17 aa, Artificial Sequence
  Description of Artificial Sequence Synthetic peptide
YFNPNSGYSTYAQKFQG

SEQ ID NO: 17 - protein, 17 aa, Artificial Sequence
  Description of Artificial Sequence Synthetic peptide
WINPNSGYSTYAQKFQG

SEQ ID NO: 18 - protein, 17 aa, Artificial Sequence
  Description of Artificial Sequence Synthetic peptide
WINPNSGYSTYSPSFQG

SEQ ID NO: 19 - protein, 17 aa, Artificial Sequence
  Description of Artificial Sequence Synthetic peptide
WINPNSGYSTYNEKFQG

SEQ ID NO: 20 - protein, 17 aa, Artificial Sequence
  Description of Artificial Sequence Synthetic peptide
YFNPNSGYSNYAQKFQG

SEQ ID NO: 21 - protein, 17 aa, Artificial Sequence
  Description of Artificial Sequence Synthetic peptide
YFNPNSGYATYAQKFQG

SEQ ID NO: 22 - protein, 17 aa, Artificial Sequence
  Description of Artificial Sequence Synthetic peptide
YFNPNSGYSTYSPSFQG

SEQ ID NO: 23 - protein, 8 aa, Artificial Sequence
  Description of Artificial Sequence Synthetic peptide
NPNSGYST

SEQ ID NO: 24 - protein, 8 aa, Artificial Sequence
  Description of Artificial Sequence Synthetic peptide
NPNSGYSN

SEQ ID NO: 25 - protein, 8 aa, Artificial Sequence
  Description of Artificial Sequence Synthetic peptide
NPNSGYAT

SEQ ID NO: 26 - protein, 10 aa, Artificial Sequence
  Description of Artificial Sequence Synthetic peptide
YFNPNSGYST

SEQ ID NO: 27 - protein, 10 aa, Artificial Sequence
  Description of Artificial Sequence Synthetic peptide
GINPNSGYST

SEQ ID NO: 28 - protein, 10 aa, Artificial Sequence
  Description of Artificial Sequence Synthetic peptide
WINPNSGYST

SEQ ID NO: 29 - protein, 10 aa, Artificial Sequence
  Description of Artificial Sequence Synthetic peptide
YFNPNSGYSN

SEQ ID NO: 30 - protein, 10 aa, Artificial Sequence
  Description of Artificial Sequence Synthetic peptide
YFNPNSGYAT

SEQ ID NO: 31 - protein, 11 aa, Artificial Sequence
  Description of Artificial Sequence Synthetic peptide
LSPGGYYVMDA

SEQ ID NO: 32 - protein, 11 aa, Artificial Sequence
  Description of Artificial Sequence Synthetic peptide
KASQNINNYLN

SEQ ID NO: 33 - protein, 11 aa, Artificial Sequence
  Description of Artificial Sequence Synthetic peptide
RASQGINNYLN

SEQ ID NO: 34 - protein, 7 aa, Artificial Sequence
  Description of Artificial Sequence Synthetic peptide
NTNNLQT

SEQ ID NO: 35 - protein, 8 aa, Artificial Sequence
  Description of Artificial Sequence Synthetic peptide
LQHNSFPT

SEQ ID NO: 36 - protein, 120 aa, Rattus norvegicus
QVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSS

SEQ ID NO: 37 - protein, 108 aa, Rattus norvegicus
DIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRT

SEQ ID NO: 38 - protein, 120 aa, Artificial Sequence [sequence omitted]

SEQ ID NO: 39 - protein, 108 aa, Artificial Sequence
  Description of Artificial Sequence Synthetic polypeptide
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRT

SEQ ID NO: 40 - protein, 328 aa, Homo sapiens
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK